(19) United States
(12) Patent Application Publication
(10) Pub. No.: US 2025/0104874 A1
(43) Pub. Date: Mar. 27, 2025

(54) METHOD FOR APPLYING LECITHIN-CHOLESTEROL ACYLTRANSFERASE (LCAT) ON HEPATOCELLULAR CARCINOMA (HCC) DIAGNOSIS, HCC TREATMENT, AND HCC RECURRENCE PREDICTION

(71) Applicant: SUN YAT-SEN UNIVERSITY CANCER CENTER (SYSUCC), GUANGZHOU (CN)

(72) Inventors: YANGXUN PAN, GUANGZHOU (CN); JIONGLIANG WANG, GUANGZHOU (CN); YAOJUN ZHANG, GUANGZHOU (CN); MINSHAN CHEN, GUANGZHOU (CN)

(21) Appl. No.: 18/673,483

(22) Filed: May 24, 2024

(30) Foreign Application Priority Data
Sep. 26, 2023 (CN) 202311252075.4

(51) Int. Cl. G16H 50/30; G16B 25/10; G16B 30/00; G16H 50/70

(52) U.S. Cl. CPC G16H 50/30; G16B 25/10; G16B 30/00; G16H 50/70

(57) ABSTRACT

A method for applying lecithin-cholesterol acyltransferase (LCAT) on hepatocellular carcinoma (HCC) diagnosis, HCC treatment, and HCC recurrence prediction is provided, including extracting a Kyoto Encyclopedia of Genes and Genomes (KEGG) metabolism-related gene data set from Gene Expression Omnibus (GEO) and processing the KEGG metabolism-related gene data set to obtain a KEGG metabolism-related gene set; integrating a data set in the GEO by a least absolute shrinkage and selection operator (LASSO) regression algorithm based on the KEGG metabolism-related gene set and constructing a risk assessment model; intersecting results obtained by performing difference analysis on a postoperative tumor of a patient undergoing hepatectomy and transcriptome sequencing data of normal tissues surrounding the postoperative tumor of the patient undergoing the hepatectomy to apply on the HCC diagnosis, the HCC treatment, and the HCC recurrence prediction in clinic.

### TECHNICAL FIELD

The present disclosure relates to a technical field of medical diagnosis, treatment, and prediction recurrence, and in particular to a method for applying lecithin-cholesterol acyltransferase (LCAT) on hepatocellular carcinoma (HCC) diagnosis, HCC treatment, and HCC recurrence prediction.

### BACKGROUND

Hepatitis B virus (HBV) may cause chronic hepatic diseases, in China, more than 90% of primary hepatocellular carcinoma (“HCC” hereinafter) are developed from hepatic cirrhosis caused by the chronic hepatic diseases. Recently, although an etiological structure of the HCC has changed, infection of the HBV is still a main cause. The HCC caused by the HBV generally undergo a change in three stages, the three stages are inflammation, hepatic cirrhosis, and HCC. Since the HCC is insidious and not easy to be sensed early in the disease, and the HCC is rapidly developed, most patients have been in a middle-and-terminal stage of the HCC when suffering from discomfort symptoms and seeing a doctor. However, treatment means for patients with the HCC are still relatively deficient, the HCC may still get worse or metastasis after the patients are treated, an overall curative effect thereof is unsatisfactory, and a median survival time of the patients with the HCC is only about 12 months.

Hepatectomy is a main means for the patients with the HCC to obtain long-term survival, and with the development of surgical techniques and related surgical instruments in recent years, more and more patients with the HCC who are previously considered as unresectable HCC patients have an opportunity to undergo the hepatectomy to realize the long-term survival.

However, even though the hepatectomy is successfully underwent, an HCC recurrence rate of the unresectable HCC patients is significantly higher that a hepatic carcinoma recurrence rate of the patients been in an early stage of the HCC. Therefore, deeply elucidating a recurrence mechanism of the HCC, screening and identifying a target gene for inhibiting tumor recurrence and development may provide the long-term survival for the unresectable HCC patients and further improve living quality thereof. A lecithin-cholesterol acyltransferase (LCAT) is one enzyme related to lipid metabolism, which widely exists in bodies of mammals, including humans, and is also the only enzyme capable of esterifying cholesterol in plasma and assisting in transporting excess cholesterol from blood and tissues to a liver, lacking the LCAT may lead to a series of metabolic-related diseases. For the HCC, LCAT low expression in HCC tissues is reported to be associated with poor tumor prognosis, so that the LCAT may play a relevant role in the development of HCC suppressor gene.

### SUMMARY

In order to solve above technical problems, the present disclosure provides a method for applying lecithin-cholesterol acyltransferase (LCAT) on hepatocellular carcinoma (HCC) diagnosis, HCC treatment, and HCC recurrence prediction to solve problems existed in the prior art.

The present disclosure provides the method for applying the LCAT on the HCC diagnosis, the HCC treatment, and the HCC recurrence prediction, including following steps.

S1: extracting a Kyoto Encyclopedia of Genes and Genomes (KEGG) metabolism-related gene data set from Gene Expression Omnibus (GEO) and processing the KEGG metabolism-related gene data set to obtain a KEGG metabolism-related gene set.

S2: integrating a data set in the GEO by a least absolute shrinkage and selection operator (LASSO) regression algorithm based on the KEGG metabolism-related gene set and constructing a risk assessment model, where constructing the risk assessment model follows one or more of a risk probability, an influence degree, and a possibility.

S3: intersecting results obtained by performing difference analysis on a postoperative tumor of a patient undergoing hepatectomy and transcriptome sequencing data of normal tissues surrounding the postoperative tumor of the patient undergoing the hepatectomy, screening and identifying the LCAT to be a high-risk recurrence gene of the patient undergoing the hepatectomy.

S4: finding that LCAT high expression is capable of activating T-lymphocyte (T) cells and natural killer (NK) cells in tumor immune microenvironment (TIME) and inhibiting tumors, and further exploring and identifying that tumor associated macrophages (TAMs) are key antigen-presenting cells (APCs) and are capable of activating immune effector cells.

S5: selecting mitogen-activated protein kinase interacting kinases (MNK) gene family for further analysis in combination with early research results, and finding that mitogen-activated protein kinase interacting kinases 1 (MNK1) has high expression in HCC tissues in combination with The Cancer Genome Atlas (TCGA) to obtain a final conclusion.

Furthermore, the S1 includes following steps.

S11: extracting KEGG metabolism-related gene data in the GEO and integrating the KEGG metabolism-related gene data to obtain the KEGG metabolism-related gene data set.

S12: diving the KEGG metabolism-related gene data set by following a ratio of 6:1:1 of a training set, a verification set, and a test set.

Compared with the prior art, the present disclosure has following beneficial effects.

The present disclosure finds that LCAT low expression in the HCC tissues in combination of the GEO is related to poor tumor prognosis of the patient undergoing the hepatectomy, and the LCAT has significant differences in the HCC tissues and normal tissues. Further, the LCAT is proved to be capable of applying on the HCC diagnosis, the HCC treatment, and the HCC recurrence prediction in clinic as a molecular marker through tissue samples of clinical HCC patients.

### BRIEF DESCRIPTION OF DRAWINGS

FIG. 1 illustrates a coefficient distribution diagram of integrating a data set in Gene Expression Omnibus (GEO) by a least absolute shrinkage and selection operator (LASSO) regression algorithm based on a Kyoto Encyclopedia of Genes and Genomes (KEGG) metabolism-related gene set and constructing a risk assessment model to screen lecithin-cholesterol acyltransferase (LCAT) to be a recurrence gene of hepatocellular carcinoma (HCC).

FIG. 2 illustrates a box plot of detecting an expression level of the LCAT in normal hepatic tissues and HCC tissues by a polymerase chain reaction (PCR) and performing statistical analysis.

FIG. 3 illustrates a histogram and statistical analysis of LCAT expression in different tumor reaction conditions (Response Evaluation Criteria in Solid Tumors, RECIST) of patients with terminal HCC undergoing hepatic artery perfusion (FOLFOX regimen) by the PCR.

FIG. 4 illustrates a Kaplan-Meier curve for analyzing Relapse-Free Survival (RFS) of patients undergoing hepatectomy according to the expression level of the LCAT, and statistical analysis for analyzing the RFS of the patients undergoing hepatectomy according to the expression level of the LCAT by Log-Rank Test.

### DETAILED DESCRIPTION

Implementations of the present disclosure are further described in detail below with reference to accompanying drawings and embodiments. The following embodiments are used to illustrate the present disclosure, but cannot be used to limit a scope of the present disclosure.

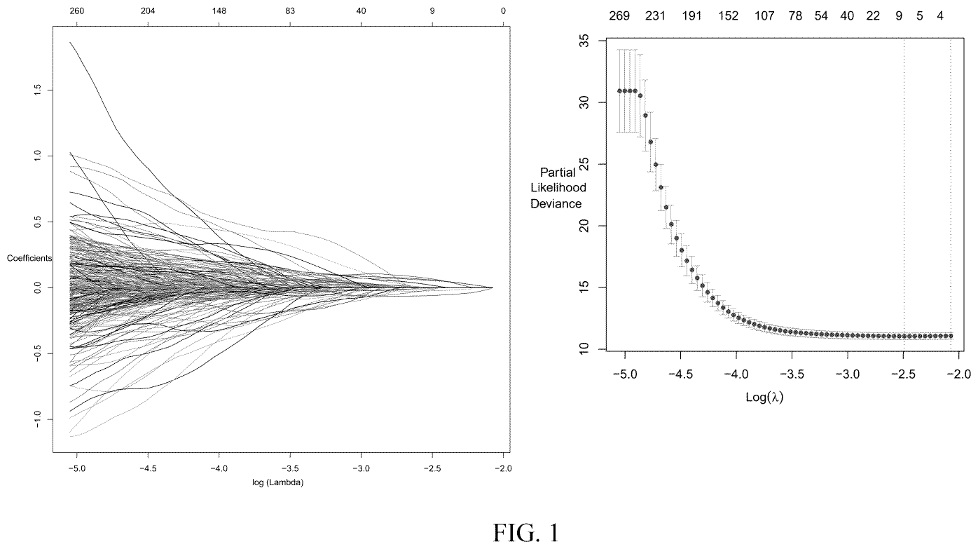

As shown in FIG. 1, the embodiments of the present disclosure provide a method for applying lecithin-cholesterol acyltransferase (LCAT) on hepatocellular carcinoma (HCC) diagnosis, HCC treatment, and HCC recurrence prediction, including following steps.

S1: extracting a Kyoto Encyclopedia of Genes and Genomes (KEGG) metabolism-related gene data set from Gene Expression Omnibus (GEO) and processing the KEGG metabolism-related gene data set to obtain a KEGG metabolism-related gene set.

S2: integrating a data set in the GEO by a least absolute shrinkage and selection operator (LASSO) regression algorithm based on the KEGG metabolism-related gene set and constructing a risk assessment model, where constructing the risk assessment model follows one or more of a risk probability, an influence degree, and a possibility.

S3: intersecting results obtained by performing difference analysis on a postoperative tumor of a patient undergoing hepatectomy and transcriptome sequencing data of normal tissues surrounding the postoperative tumor of the patient undergoing the hepatectomy, screening and identifying the LCAT to be a high-risk recurrence gene of the patient undergoing the hepatectomy.

S4: finding that LCAT high expression is capable of activating T-lymphocyte (T) cells and natural killer (NK) cells in tumor immune microenvironment (TIME) and inhibiting tumors, and further exploring and identifying that tumor associated macrophages (TAMs) are key antigen-presenting cells (APCs) and are capable of activating immune effector cells.

S5: selecting mitogen-activated protein kinase interacting kinases (MNK) gene family for further analysis in combination with early research results, and finding that mitogen-activated protein kinase interacting kinases 1 (MNK1) has high expression in HCC tissues in combination with The Cancer Genome Atlas (TCGA) to obtain a final conclusion.

Based above, it can be knew that LCAT low expression in the HCC tissues in combination of the GEO is related to poor tumor prognosis of the patient undergoing the hepatectomy, and the LCAT has significant differences in the HCC tissues and normal tissues.

Based on above research basis, it can propose that for patients with the HCC, the LCAT is proved to be capable of applying on the HCC diagnosis, the HCC treatment, and the HCC recurrence prediction in clinic as a molecular marker through tissue samples of clinical HCC patient.

Specifically, the LASSO regression algorithm is to minimize a sum of squares of residual errors under constraint conditions that a sum of absolute values of regression coefficients is less than a constant, so that some regression coefficients strictly equal to 0 is generated to obtain an interpretable model, and a mathematical expression thereof is as follows:

$${B}_{L A S S O} = {arg}_{B} min \{ \vert Y - {\sum }_{j = 1}^{p} {X}_{j} {B}_{j} \vert \}$$

$$s . t . {\sum }_{j = 1}^{p} \vert {B}_{j} \vert \leq t$$

Specifically, the t is greater than 0 and is an adjustment parameter, and an overall regression coefficient is compressed by controlling the adjustment parameter t. Value determination of the t is estimated by cross-validation proposed by Efron and Tibshirrani (1993). The mathematical expression is also equivalent to minimizing following penalty least squares method:

$${B}_{LASSO} = {arg}_{B} min \{ {\vert Y - {\sum }_{j = 1}^{p} {X}_{j} {B}_{j} \vert}^{2} + a {\sum }_{j = 1}^{p} \vert {B}_{j} \vert \}$$

Specifically, the a and the t correspond to each other on a one-to-one basis, and may be converted from each other. A main advantage of the LASSO regression algorithm is that compression on variables with larger parameter estimation is small, and compression on variables with smaller parameter estimation is 0, and the parameter estimation of the LASSO regression algorithm has continuity and is suitable for model selection of high-dimensional data. Tibshirrani proposes a Fused Lasso method in 2005, which satisfies sparsity of model coefficients and coefficient differences, so that adjacent coefficients are smoother.

Based on above, under a condition that a sum of absolute values of model coefficients of the LASSO regression algorithm is less than one constant, the sum of the squares of the residual errors is minimized, an effect in an aspect of variable selection is better than that of gradual regression, principal components regression, ridge regression, partial least squares, etc., so that defects of conventional methods in model selection are well overcome, and the risk assessment model is accurately constructed

Specifically, constructing the risk assessment model follows one or more of the risk probability, the influence degree, and the possibility, following performs matching on principles that are followed for constructing the risk assessment model, and further perform the HCC diagnosis, the HCC treatment, and the HCC recurrence prediction to obtained following table:

| | constructed | total effective |
|---|---|---|
| | model (%) | rate (%) |
| risk probability | 70.6 | 62.8 |
| influence degree | 70.9 | 62.4 |
| possibility | 71.2 | 62.7 |
| risk probability & influence | 78.5 | 67.5 |
| degree | | |
| risk probability & possibility | 78.1 | 67.2 |
| influence degree & possibility | 78.8 | 68.0 |
| risk probability & influence | 86.4 | 72.8 |
| degree & possibility | | |
| matched group | 60.5 | 50.4 |

It can be seen from the above table that a model constructing effect and an application effect of the LCAT on the HCC diagnosis, the HCC treatment, and the HCC recurrence prediction are better.

Following specific experiments are performed for applying the LCAT on the HCC diagnosis, the HCC treatment, and the HCC recurrence prediction.

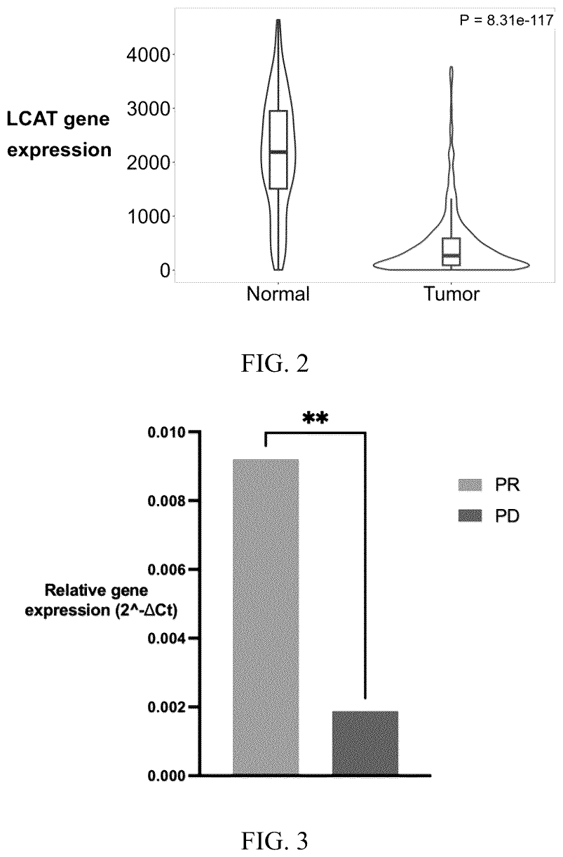

A specific experiment for applying the LCAT on the HCC diagnosis is as follows. 100 cases of tissue samples, including 50 cases of HCC tissues and 50 cases of normal hepatic tissues, are selected for experimental analysis, where LCAT expression quantities of the 100 cases of the tissue samples are normalized by Log 2 (TPM+1) and then displayed in forms of violin graphs in the figure, and the violin graphs formed by different LCAT expression quantities of different tissue groups are respectively put at a left side and a right side. A violin graph of the HCC tissues is on the right side, and a violin graph of the normal hepatic tissues is on the left side, and an analysis result is shown in FIG. 2.

As shown in FIG. 2, LCAT expression quantities of the HCC tissues are obviously lower than LCAT expression quantities of the normal hepatic tissues, and above results show that the LCAT expression quantities of the HCC tissues are obviously lower than the LCAT expression quantities of the normal hepatic tissues, so that the LCAT expression quantities are capable of assisting in the HCC diagnosis as the molecular marker.

A specific experiment for applying the LCAT on the HCC treatment is as follows.

Inventors of the present disclosure find that in patients with terminal HCC undergoing hepatic artery perfusion (FOLFOX regimen), the LCAT expression quantities significantly affect a curative effect of treatment, and an overall evaluation of patients with high LCAT expression quantities on a treatment reaction is significantly better than an overall evaluation of patients with low LCAT expression quantity. A specific method for applying the LCAT on the HCC treatment includes extracting ribonucleic acid (RNA) of puncture specimen tissues of the patients obtained before undergoing the hepatic artery perfusion; dividing RNA sequencing into a progressive disease (PD) group and a partial response (PR) group according to different tumor reaction conditions (Response Evaluation Criteria in Solid Tumors, RECIST) of patients with terminal HCC undergoing the hepatic artery perfusion; and comparing difference between clinical data and messenger RNA (mRNA) levels of the LCAT in tumor tissues of patients in the PD group and patients in the PR group. Normalized LCAT expression quantities of the patients in the PD group and the patients in the PR group are presented in a histogram, and a result thereof is shown in FIG. 3.

As shown in FIG. 3, the normalized LCAT expression quantities of the patients in the PD group and the patients in the PR group are significantly different, LCAT expression quantities of the patients in the PR group is significantly higher than LCAT expression quantities of the patients in the PD group. The above result indicates that in the patients with the terminal HCC undergoing the hepatic artery perfusion, a treatment effect of patients with high LCAT expression quantities is more significant.

A specific experiment for applying the LCAT on the HCC diagnosis, the HCC treatment, and the HCC recurrence prediction is as follows.

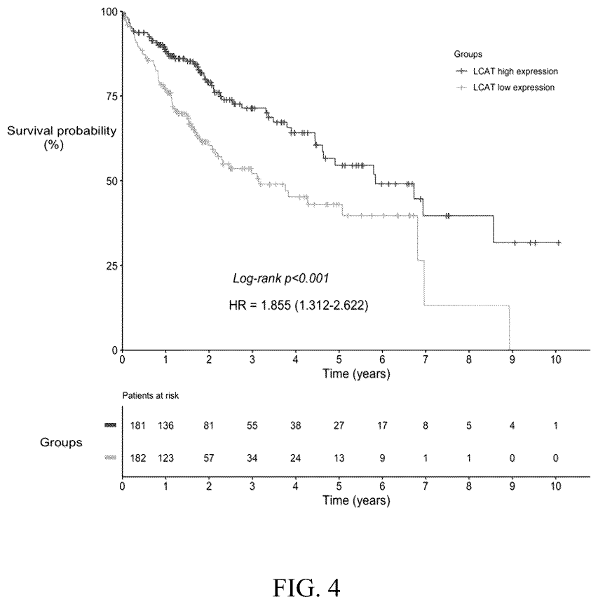

A method for analyzing a correlation between LCAT expression quantities and prognosis of the patients undergoing the hepatectomy includes selecting 363 cases of the patients undergoing the hepatectomy to detect the LCAT expression quantities, taking a median of the LCAT expression quantities as a boundary, dividing the 363 cases of the patients undergoing the hepatectomy into an LCAT high expression group and an LCAT low expression group, and analyzing a relationship between the LCAT expression quantities and a Relapse-Free Survival (RFS) of the patients undergoing the hepatectomy by R to obtain FIG. 4.

As shown in FIG. 4, a statistical analysis result shows that the LCAT expression quantities are significantly positively correlated with the RFS of the patients undergoing the hepatectomy (p<0.001), and it is clearly indicated that patients with high LCAT expression quantities have better prognosis. Therefore, the LCAT expression quantities may be a potential molecular marker for predicting the prognosis of the patients undergoing the hepatectomy.

While embodiments of the present disclosure have been shown and described for purposes of illustration and description, it is to be understood that the above embodiments are exemplary and should not be construed as limiting the present disclosure, and those who skilled in the art may make changes, modifications, substitutions, and variations to the above embodiments within a scope of the present disclosure.

What is claimed is:

1. A method for applying lecithin-cholesterol acyltransferase (LCAT) on hepatocellular carcinoma (HCC) recurrence prediction, including:
S1: extracting a Kyoto Encyclopedia of Genes and Genomes (KEGG) metabolism-related gene data set from Gene Expression Omnibus (GEO) and processing the KEGG metabolism-related gene data set to obtain a KEGG metabolism-related gene set;
S2: integrating a data set in the GEO by a least absolute shrinkage and selection operator (LASSO) regression algorithm based on the KEGG metabolism-related gene set and constructing a risk assessment model, wherein constructing the risk assessment model follows one or more of a risk probability, an influence degree, and a possibility;
S3: intersecting results obtained by performing difference analysis on a postoperative tumor of a patient undergoing hepatectomy and transcriptome sequencing data of normal tissues surrounding the postoperative tumor of the patient undergoing the hepatectomy, screening and identifying the LCAT to be a high-risk recurrence gene of the patient undergoing the hepatectomy;
S4: finding that LCAT high expression is capable of activating T-lymphocyte (T) cells and natural killer (NK) cells in tumor immune microenvironment (TIME) and inhibiting tumors, and further exploring and identifying that tumor associated macrophages (TAMs) are key antigen-presenting cells (APCs) and are capable of activating immune effector cells; and
S5: selecting mitogen-activated protein kinase interacting kinases (MNK) gene family for further analysis in combination with early research results, and finding that mitogen-activated protein kinase interacting kinases 1 (MNK1) has high expression in HCC tissues in combination with The Cancer Genome Atlas (TCGA) to obtain a final conclusion.

2. The method for applying the LCAT on the HCC recurrence prediction according to claim 1, wherein the S1 comprises:
S11: extracting KEGG metabolism-related gene data in the GEO and integrating the KEGG metabolism-related gene data to obtain the KEGG metabolism-related gene data set; and
S12: diving the KEGG metabolism-related gene data set by following a ratio of 6:1:1 of a training set, a verification set, and a test set.
